(19) United States
(12) Patent Application Publication
(10) Pub. No.: US 2026/0158205 A1
(43) Pub. Date: Jun. 11, 2026

(54) FLUID DISTRIBUTION SYSTEM

(71) Applicant: ELATE MEDICAL, INC., Houston, TX (US)

(72) Inventor: Moonsoo Martin Jin, Houston, TX (US)

(21) Appl. No.: 18/706,643

(22) PCT Filed: Jan. 2, 2024
(86) PCT No.: PCT/IB2024/050013

§ 371 Date: May 1, 2024

(30) Foreign Application Priority Data
Jan. 6, 2023 (KR) 10-2023-0002257

(51) Int. Cl. A61M 1/36

(52) U.S. Cl. CPC A61M 1/3693

(57) ABSTRACT

Provided is a fluid distribution system including: a main body; a plate-shaped main body cover formed to cover at least part of an upper portion of the main body; and a plurality of selectors arranged on the main body cover to be spaced apart from each other and to make fluid communication with each other, wherein one selector includes: a cylindrical upper case including a first port extending from a center of an upper surface to an outer circumferential surface of an upper surface on the upper surface and a plurality of third upper holes arranged along the outer circumferential surface of the upper surface at equal intervals and having an open lower surface; a cylindrical lower case having at least part inserted into the upper case through a lower surface of the upper case; and a main body coupling portion protruding downwards from a bottom surface of the lower case and coupled to the main body.

### BACKGROUND OF THE INVENTION

### 1. Field of the Invention

The present disclosure relates to a fluid distribution system, and more specifically, to a cell handling system in which complex lines can be simplified and which is easy to operate.

### 2. Description of the Related Art

Content described in this part simply provides background information about the present disclosure and does not constitute the related art.

Treatment that uses T cells by separating the T cells from body fluids of cancer patients, such as blood or cancer tissue, has been spotlighted among treatments for cancer patients. Physical and chemical methods are used to separate specific cells from the body fluids of cancer patients, but the most effective method is centrifugation, which requires cell handling in advance.

FIG. 9 illustrates a fluid distribution system for separating specific cells from a mixture. Mixtures or other compounds to be separated are accommodated in an infusion bag of FIG. 9, and each infusion bag is connected to one or more conduits. One or more two-way valves (cylinders numbered in FIG. 9) for opening and closing each connected conduit are arranged in the fluid distribution system. By controlling the opening and closing of one or more valves, a path of the mixture or compound flowing through a specific infusion bag can be adjusted.

Meanwhile, in the above-described system, one or more valves are required to be arranged at each branch point of the conduit. As shown in FIG. 9, the path of fluid flow can be controlled by operating each of 24 valves. As a result, it is difficult to operate the system, and internal line configuration is complicated, and the risk of failure is high.

(Patent Document 1) KR 10-2005-0103604 A

### SUMMARY OF THE INVENTION

The present disclosure is devised to solve the above problems.

The present disclosure provides a fluid distribution system in which a plurality of flow paths can be operated by using one selector.

The present disclosure also provides a fluid distribution system which is easy to operate and has a simple line configuration and low risk of failure.

The present disclosure also provides a fluid distribution system that intuitively allows a user to easily reconfigure a plurality of flow paths.

Objectives to be solved by the present disclosure are not limited to the objectives mentioned above, and other unmentioned objectives will be clearly understood from the description below.

According to an aspect of the present disclosure, there is provided a fluid distribution system including: a main body; a plate-shaped main body cover formed to cover at least part of an upper portion of the main body; and a plurality of selectors arranged on the main body cover to be spaced apart from each other and to make fluid communication with each other, wherein one selector includes: a cylindrical upper case including a first port extending from a center of an upper surface to an outer circumferential surface of the upper case on the upper surface and a plurality of third upper holes arranged along the outer circumferential surface of the upper case at equal intervals and having an open lower surface; a cylindrical lower case having at least part inserted into the upper case through the lower surface of the upper case; and a main body coupling portion protruding downwards from a bottom surface of the lower case and coupled to the main body.

The upper case may include: a first upper hole formed on one surface of the first port formed in parallel to an outer circumferential surface of the upper case; a second upper hole formed in a center of the upper surface of the upper case and passing through an interior ceiling surface of the upper case from the upper surface of the upper case; and an upper flow path extending from the first upper hole to the second upper hole and formed inside the first port.

The lower case may include: a first transfer hole formed in an upper surface of the lower case and facing the second upper hole; the second transfer hole formed in an outer circumferential surface of the lower case and facing one of the plurality of third upper holes; and a transfer flow path extending from the first transfer hole to the second transfer hole and formed inside the lower case.

The main body coupling portion may include: a coupling groove formed toward an inside from a bottom surface of the main body coupling portion; a position adjustment protrusion arranged on an outer circumferential surface of the main body coupling portion; and a zero-point adjustment protrusion arranged on the outer circumferential surface of the main body coupling portion, wherein a distance between the bottom surface of the main body coupling portion and an end of the position adjustment protrusion is greater than a distance between the bottom surface of the main body coupling portion and an end of the zero-point adjustment protrusion.

A number of the plurality of third upper holes may be n (where n is a natural number), one zero-point adjustment protrusion may be arranged, and (n−1) position adjustment protrusions may be arranged, and the zero-point adjustment protrusion and the position adjustment protrusion may be arranged at equal intervals.

A center of the zero-point adjustment protrusion may be aligned with a center of the second transfer hole.

The main body may include: a selector installation portion into which the main body coupling portion is inserted; a power transmission portion formed in a center of the selector installation portion and inserted into the coupling groove; a zero-point adjustment sensor arranged on an inner circumferential surface of the selector installation portion and formed to face one end of the zero-point adjustment protrusion; and a position adjustment sensor arranged on the inner circumferential surface of the selector installation portion and formed to face one end of one of the position adjustment protrusions.

The main body may include: a motor arranged inside of the main body and configured to rotate the main body coupling portion; and a manipulation portion configured to adjust a rotation angle and rotation speed of the motor.

The main body cover may include a magnet, and the main body may further include a metal material coupled to the magnet.

The main body may further include a plurality of guide shafts arranged on an upper surface of the main body, and the main body cover may include a plurality of guide holes through which the plurality of guide shafts pass so that the main body cover is coupled to the main body in a preset position.

The fluid distribution system may further include lines configured to connect at least one of a plurality of third upper holes of one among the plurality of selectors to a first upper hole of another one among the plurality of selectors.

The fluid distribution system may further include a pump, wherein the lines are connected to the pump.

As described above, according to one or more embodiments of the present disclosure, a fluid distribution system can replace a plurality of valves by using only one selector, and the use of the fluid distribution system is simple, and the effect of cost reduction is excellent.

Also, when a plurality of selectors according to the present disclosure are used, tubing in a variety of number of cases is possible, which has the effect of allowing a user to form various flow paths in a desired combination method.

Additionally, according to another embodiment of the present disclosure, since at least a portion of the selector is made of a transparent material, the flow of fluid can be seen at a glance.

### BRIEF DESCRIPTION OF THE DRAWINGS

FIG. 1 is a front perspective view of a fluid distribution system according to an embodiment of the present disclosure;

FIG. 2 is an exploded perspective view of a fluid distribution system according to an embodiment of the present disclosure;

FIG. 3 is an enlarged view of a separated state of a main body cover and part of a selector installation portion according to an embodiment of the present disclosure;

FIG. 4 is an exploded perspective view of a selector according to an embodiment of the present disclosure;

FIG. 5 is an enlarged view of bottom and part of the bottom of the main body cover to which the selector is coupled, according to an embodiment of the present disclosure;

FIG. 6 shows a cross-section of a selector according to an embodiment of the present disclosure;

FIGS. 7 and 8 are diagrams for describing a method of operating a fluid distribution system according to another embodiment of the present disclosure; and

FIG. 9 shows a fluid distribution system according to the related art.

### DETAILED DESCRIPTION OF THE INVENTION

Hereinafter, some embodiments of the present disclosure will be described in detail with reference to exemplary drawings. When adding reference numerals to components in each drawing, it should be noted that the same components are given the same reference numerals as much as possible even if they are shown in different drawings. Additionally, in describing the present disclosure, if it is determined that a detailed description of a related known configuration or function may obscure the gist of the present disclosure, the detailed description will be omitted.

In describing the components of the embodiment according to the present disclosure, symbols such as first, second, i), ii), a), and b) may be used. These symbols are only used to distinguish the component from other components, and the nature, sequence, or order of the component is not limited by the symbol. In the specification, when a portion is said to “include” or “have” a certain element, this means that it does not exclude other elements, but may further include other elements, unless explicitly stated to the contrary.

In the present disclosure, “upward” refers to a direction in which a main body 10 is raised in a height direction.

Also, in the present disclosure, “downward” refers to a direction in which the main body 10 is lowered in the height direction.

Also, in the present disclosure, “zero-point” refers to a position of the lower case 24 when the center line of a second transfer hole 244 of a lower case 24 and the center line of a reference third upper hole of an upper case 22 coincide.

Also, in the present disclosure, “interval” may refer to a straight-line distance or a distance measured along the surface of a curved surface. Alternatively, the interval may be a rotation angle measured based on the center of a virtual circle.

Also, in the present disclosure, some components of a fluid distribution system 1 are omitted. For example, conduits connecting the fluid distribution system 1 to the outside are omitted, but it will be noted that the fluid distribution system 1 may be in fluid communication with the outside.

Also, in the present disclosure, eight third upper holes 228 are formed in one selector 20. However, this is just an example, and it will be noted that the number of third upper holes 228 may be adjusted if necessary. For example, three or sixteen third upper holes 228 may be formed in one selector 20 of the present disclosure.

Hereinafter, the fluid distribution system 1 according to the present disclosure will be described with reference to the drawings.

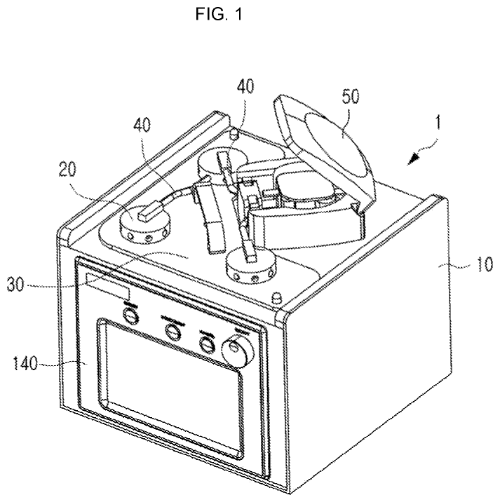

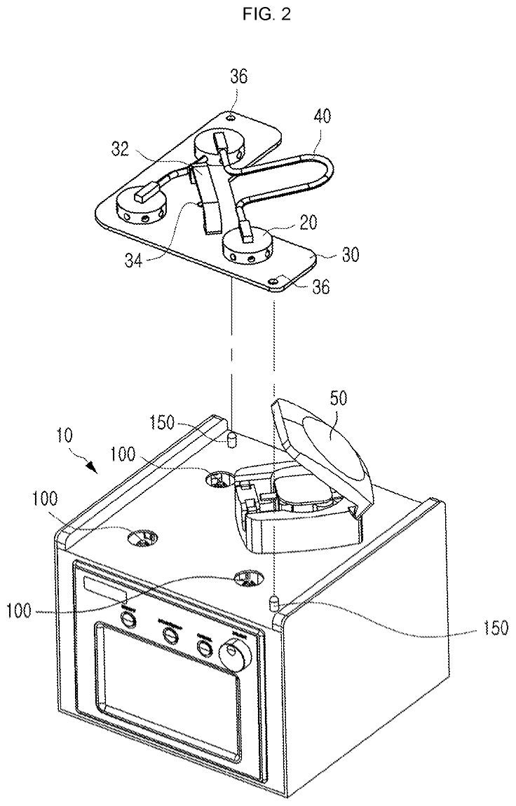

FIG. 1 is a front perspective view of a fluid distribution system according to an embodiment of the present disclosure. FIG. 2 is an exploded perspective view of a fluid distribution system according to an embodiment of the present disclosure.

Referring to FIGS. 1 and 2, the fluid distribution system 1 according to an embodiment of the present disclosure includes a main body 10, a selector 20, a main body cover 30, lines 40, and a pump 50.

The main body 10 electrically operates, and is configured to adjust a path of fluid flowing from the outside. Preferably, the main body 10 has a hexagonal shape, but the present disclosure is not limited thereto.

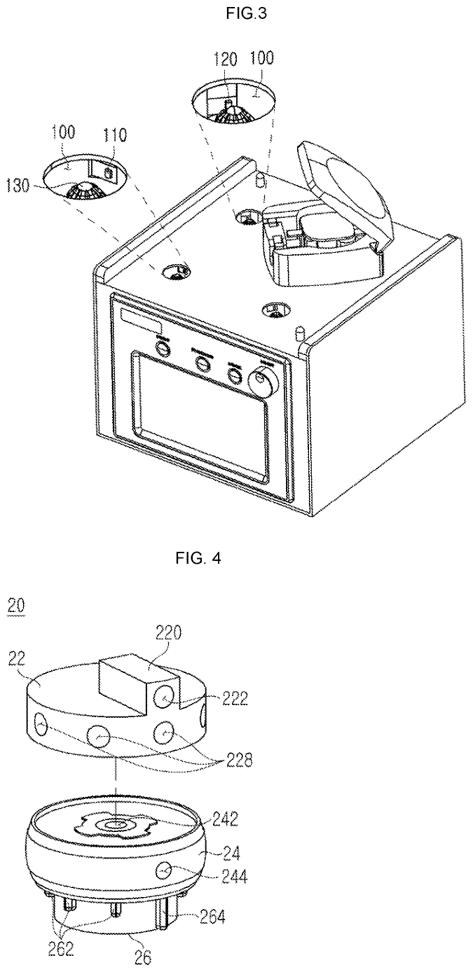

The main body 10 includes a selector installation portion 100, a position adjustment sensor (see 110 of FIG. 3), a zero-point adjustment sensor (see 120 of FIG. 3), a power transmission portion (see 130 of FIG. 3), a manipulation portion 140, and a guide shaft 150. The position adjustment sensor 110, the zero-point adjustment sensor 120, and the power transmission portion 130 will be described in detail with reference to FIG. 3. The selector installation portion 100 is formed so that at least part of the selector 20 is inserted into the selector installation portion 100, and is formed toward the inside of the main body 10 from an upper surface of the main body 10. The selector installation portion 100 is formed to correspond to the shape of the selector 20, and in the present disclosure, the selector 20 has a cylindrical shape and thus, preferably, the selector installation portion 100 has a cylindrical shape.

The manipulation portion 140 is provided at one side of the main body 10 and may include a display or speaker through which values sensed by the position adjustment sensor 110 and the zero-point adjustment sensor 120 are output. The user may check the values displayed on the manipulation portion 140 and may adjust alignment of the selector 20.

Meanwhile, the alignment of the selector 20 may be performed by a motor (not shown) arranged inside the main body 10. Electrical signals for adjusting the rotation angle and rotation speed of the motor may be input through the manipulation portion 140 so that alignment of the selector 20 may be adjusted. Alternatively, alignment of the selector 20 may be directly controlled by the user. Specifically, the user may manually control rotation of internal configuration of the selector 20, and in this case, preferably, at least part of the selector 20 may be formed of a transparent material. Also, the selector 20 may be marked with a scale for checking the zero-point. The user may control the rotation of the internal configuration of the selector 20 to a correct position by looking at the scale.

The guide shaft 150 is inserted into the guide hole 36 so that coupling of the main body cover 30 and the main body 10 becomes easy.

A plurality of selectors 20 are arranged on the upper surface of the main body 10. The selectors are configured to primarily accommodate the fluid flowing from the outside and to transfer the flowing fluid to the pump 50 or another selector 20. Part of the selector 20 is configured to rotate. Thus, the direction of the flow path may be determined. In this case, rotation of the selector 20 may be performed by the motor provided at the main body 10 or directly by the user.

One selector 20 and another selector 20 may be connected to each other via the lines 40. Meanwhile, a detailed structure of the selector 20 will be described in detail with reference to FIGS. 4 through 6.

The main body cover 30 is coupled to the main body 10 between the selector 20 and the main body 10 when the selector 20 is mounted on the main body 10. Preferably, the main body cover 30 is of roughly a plate type so as to be easily mounted on the main body 10.

The main body cover 30 includes a handle 32, a magnet 34, and a guide hole 36. The handle 32 is provided at the upper surface of the main body cover 30, and the user grasps the handle 32 so that detachment of the main body cover 30 and the main body 10 may be easily performed.

The magnet 34 is provided at one side of the main body cover 30, and coupling of the main body cover 30 and the main body 10 may be easily performed through an interaction between the magnet 34 and the main body 10. In this case, a member (not shown) for interacting with the magnet 34 may be further included in the inside of the main body 10. For example, the member for interacting with the main body 10 may be a magnet arranged so that the polarity of one side of the member is opposite to the polarity formed on one side of the magnet 34. Alternatively, the member for interaction may be a metal material for interaction with the magnet 34.

The guide hole 36 is formed so that the guide shaft 150 may be inserted into the guide hole 36. The guide hole 36 is preferably formed close to the corner of the main body cover 30 so as not to interfere with the interconnection of the plurality of selectors 20, the lines 40, and the pump 50 on the main body cover 30.

Also, it is preferable that two or more guide holes 36 and guide shafts 150 are each provided. Thus, both may be coupled to each other in an exact position in which the main body cover 30 is required to be coupled to the main body 10. The pump 50 is configured to transfer fluid flowing from one of the plurality of selectors 20 to another direction. The pump 50 is sufficient as a device for transporting liquid, and any one of known pumps may be selected.

FIG. 3 is an enlarged view of a separated state of a main body cover and part of a selector installation portion according to an embodiment of the present disclosure.

Referring to FIG. 3, preferably, the selector installation portion 100 has a shape of a groove so that at least part of the selector 20 is inserted into the selector installation portion 100. The selector installation portion 100 is formed at the main body 10 as many as the required number of selectors 20.

The position adjustment sensor 110, the zero-point adjustment sensor 120, and the power transmission portion 130 are arranged in the selector installation portion 100. The position adjustment sensor 110 and the zero-point adjustment sensor 120 are arranged on an inner circumferential surface of the selector installation portion 100, and the position adjustment sensor 110 is arranged in a higher position from the bottom surface of the selector installation portion 100 than the zero-point adjustment sensor 120. This is due to the structure of the selector 20, and this will be described in detail with reference to FIG. 4.

The position adjustment sensor 110 and the zero-point adjustment sensor 120 are configured to recognize ends of protrusions (see 262 and 264 of FIG. 4) of the selector 20. The position adjustment sensor 110 and the zero-point adjustment sensor 120 may be any type of a mechanical type, an electric type, a magnetic type or an optical type. Also, it may be any type of contact sensor or non-contact sensor.

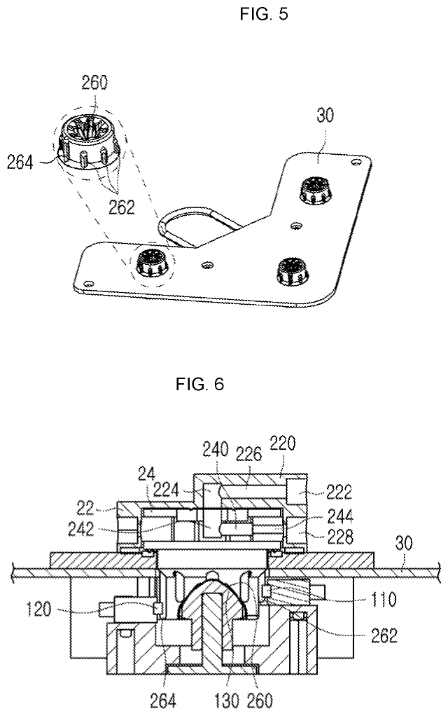

The power transmission portion 130 is configured to transfer power generated from a motor (not shown) arranged inside the main body 10, i.e., rotational torque, to the selector 20. Preferably, the power transmission portion 130 has a shape protruding to be coupled to a coupling groove (see 260 of FIG. 5) of the selector 20. In this case, preferably, the coupling groove 260 has a shape corresponding to the shape of the power transmission portion 130.

Also, the power transmission portion 130 may include an uneven portion formed on an outer circumferential surface thereof, and the coupling groove 260 has a shape corresponding thereto and thus, both may be engaged with each other and coupled to each other. Thus, when the power transmission portion 130 is coupled to the coupling groove 260, the power transmission portion 130 may be coupled in a correct position of the coupling groove 260. Also, regardless of which direction the power transmission unit 130 rotates, the selector 20 may be rotated by a correct rotation angle.

FIG. 4 is an exploded perspective view of a selector according to an embodiment of the present disclosure. FIG. 5 is an enlarged view of bottom and part of the bottom of the main body cover to which the selector is coupled, according to an embodiment of the present disclosure. FIG. 6 shows a cross-section of a selector according to an embodiment of the present disclosure.

Meanwhile, in FIG. 6, the upper case 22 and the lower case 24 are coupled to each other via a predetermined gap therebetween, but this is exaggerated for understanding, and it should be understood that the upper case 22 and the lower case 24 are coupled to each other without any gap. To this end, the lower case 24 may be press-fitted into the upper case 22, or a sealing device (not shown) may be further provided between the upper case 22 and the lower case 24.

Referring to FIGS. 4 through 6, the selector 20 according to an embodiment of the present disclosure includes an upper case 22, a lower case 24, and a main body coupling portion 26.

The upper case 22 may accommodate the fluid flowing into the selector 20 primarily. The upper case 22 may have roughly a cylindrical shape, but the present disclosure is not limited thereto.

A first port 220 that extends from the center of an upper surface to the outer circumferential surface of the upper case 22, is arranged on the upper surface of the upper case 22. An upper flow path (see 226 of FIG. 6) is formed inside the first port 220. The upper flow path 226 extends between a first upper hole 222 and a second upper hole (see 224 of FIG. 6) and is configured to transfer the fluid flowing from a conduit connected to the first port 220 to the lower case 24.

Meanwhile, the first upper hole 222 is formed in one surface formed in parallel to the outer circumferential surface of the upper case 22, as one surface of the first port 220. The second upper hole 224 is formed to an interior ceiling surface of the upper case 22. Since the upper flow path 226 extends from the first upper hole 222 to the second upper hole 224, the fluid flowing from an outside of the upper case 22 may be transferred to the inside of the upper case 22.

A plurality of third upper holes 228 are formed on a side surface, i.e., an outer circumferential surface of the upper case 22. In this case, the plurality of third upper holes 228 are preferably arranged at equal intervals along the outer circumferential surface of the upper case 22. The fluid flowing into the inside of the upper case 22 may be discharged to the outside of the upper case 22 through the plurality of third upper holes 228. If eight third upper holes 228 are formed in the outer circumferential surface of the upper case 22, the fluid may be bypassed to eight paths by one selector 20. Thus, the role played by eight valves installed in each of eight paths can be solved with just one selector 20 of the present disclosure, reducing production costs, simplifying line configuration, and lowering the risk of failure of the entire system.

Each of the third upper holes 228 may have a circular shape and preferably, may have the same size and same shape as those of the first upper hole 222 formed in the first port 220. In this case, one of the plurality of third upper holes 228 is arranged exactly on a straight line with the center of the first upper hole 222. The third upper hole 228 is referred to as a “reference third upper hole”.

A plurality of selectors 20 may be connected to each other via the third upper hole 228 and the first upper hole 222. Specifically, one third upper hole among three upper holes 228 of one of the plurality of selectors 20 may be connected to the first upper hole 222 of another one selector 20 among the plurality of selectors 20.

The lower case 24 may accommodate the fluid flowing into the selector 20 secondarily. Also, the lower case 24 plays the most important role in selecting the path of the fluid introduced through the first upper hole 222. To this end, the lower case 24 is inserted into the upper case 22, and a transfer path 240 is formed inside the lower case 24. The lower case 24 may have a cylindrical shape, and the present disclosure is not necessarily limited thereto.

The transfer path 240 extends between the first transfer hole 242 and the second transfer 244. The first transfer hole 242 is formed in an upper surface of the lower case 24 and is formed to face the second upper hole 224. In this case, the first transfer hole 242 and 224 are circular holes having the same size, and when the lower case 24 is fully inserted into the upper case 22, the center of the first transfer hole 242 and the center of the second transfer hole 224 exactly coincide with each other. Thus, the fluid flowing through the first port 220 may leak to the outside of the lower case 24.

The second transfer hole 244 is formed in a side surface, i.e., a circumferential surface of the lower case 24, to face the third upper hole 228. In this case, the second transfer hole 244 and the third upper hole 228 are circular holes having the same size, and when the lower case 24 is fully inserted into the upper case 22, the center of the second transfer hole 244 and the center of the third upper hole 228 exactly coincide with each other. Thus, the fluid transferred through the second transfer hole 244 may not leak to the outside of the lower case 24.

As the lower case 24 rotates, the third upper hole 228 that the second transfer hole 244 faces, varies. When the second transfer hole 244 faces the reference third upper hole, the fluid flowing into the first port 220 is discharged to a conduit connected to the reference third upper hole. If the second transfer hole 244 faces another third upper hole of the plurality of third upper holes 228, the fluid will be discharged to the conduit connected to the third upper hole.

In other words, according to an embodiment of the present disclosure, the fluid flowing through the first upper hole 222 sequentially passes through the upper flow path 260 and the transfer flow path 240 and is discharged through the third upper hole 228. Meanwhile, opposite directions are also possible. That is, the fluid flowing through one third upper hole 228 may sequentially pass through the transfer flow path 240 and the upper flow path 226 and may be discharged through the first upper hole 222. The flow order of the fluid may be design-changed according to the role and arrangement of the selector 20. In other words, when a plurality of selectors 20 according to an embodiment of the present disclosure are connected to each other, flow from the first port 220 to the second transfer hole 244 is formed in one direction and the flow is changed in an opposite direction to one direction, flow from the second transfer hole 244 to the first port 220 will be formed. In this case, conversion of flow may be performed by reversing an input/output direction of a pump and a connection direction of lines 40 according to the present disclosure.

The main body coupling portion 26 protrudes downwards from the bottom surface of the lower case 24, and the selector 20 may be coupled to the main body 10 through the main body coupling portion 26. The main body coupling portion 26 may be formed integrally with the lower case 24 or may be separated from each other. Also, the main body coupling portion 26 may have an overall cylindrical shape.

The main body coupling portion 26 may transfer a rotational torque of the motor transferred by the power transmission portion 130 to the lower case 24. That is, the rotational torque generated in the motor is transferred to the lower case 24 through the power transmission portion 130 and the main body coupling portion 26, and the lower case 24 rotates inside the upper case 22. In this case, the upper case 22 does not rotate in a state in which it is coupled to the main body cover 30.

The main body coupling portion 26 includes a coupling groove 260 and all or portions of a position adjustment protrusion 262 and a zero-point adjustment protrusion 264 (see FIG. 5).

The coupling groove 260 is formed toward an inside of the main body coupling portion 26 from the bottom surface of the main body coupling portion 26 so that the power transmission portion 130 may be inserted into the coupling groove 260. The coupling groove 260 is preferably formed to correspond to the shape of the power transmission portion 130.

One or more position adjustment protrusions 262 are arranged along the outer circumferential surface of the main body coupling portion 26.

One zero-point adjustment protrusion 264 is arranged on the outer circumferential surface of the main body coupling portion 26.

Meanwhile, the number of position adjustment protrusions 262 is 1 less than the number of third upper holes 228. Also, since the number of zero-point adjustment protrusions 264 is one, the sum of the number of position adjustment protrusions 262 and the number of zero-point adjustment protrusions 264 is identical to the number of third upper holes 228. For example, when the number of third upper holes 228 is eight, the number of position adjustment protrusions 262 is seven, and the number of zero-point adjustment protrusions 264 is one. In this case, the position adjustment protrusion 262 and the zero-point adjustment protrusion 264 are arranged at equal intervals.

A distance between the bottom surface of the main body coupling portion 26 and a lower end of the position adjustment protrusion 262 is greater than a distance between the bottom surface of the main body coupling portion 26 and a lower end of the zero-point adjustment protrusion 264. That is, based on the bottom of the main body coupling portion 26, the end of the position adjustment protrusion 262 is located higher than the end of the zero-point adjustment protrusion 264 (see FIG. 6). Thus, the zero-point adjustment sensor 120 may sense only the zero-point adjustment protrusion 264.

By using the zero-point adjustment protrusion 264, the lower case 24 may be adjusted so that the lower case 24 is located at the zero-point. The zero-point adjustment protrusion 264 faces the zero-point adjustment sensor 120 when the lower case 24 is located at the zero-point, i.e., when the center of the reference third upper hole 228 of the upper case 22 and the center of the second transfer hole 244 coincide with each other.

In this case, since the zero-point adjustment sensor 120 senses only the end of the zero-point adjustment protrusion 264, the zero-point adjustment sensor 120 does not sense the position adjustment protrusion 262 but senses only the zero-point adjustment protrusion 264. If the zero adjustment protrusion 264 is not sensed, it means that the lower case 24 is located at the zero-point. In this case, the motor may be controlled so that the lower case 24 is located at the zero-point.

The position adjustment sensor 110 is configured to sense the end of the position adjustment protrusion 262. In this case, one or more position adjustment sensors 110 may be arranged on an inner circumferential surface of the selector installation portion 100, and the number of position adjustment sensors 110 may be different according to design matters.

The position adjustment sensor 110 is spaced apart from the zero-point adjustment sensor 120 by a predetermined distance. Here, the predetermined distance (angle) is an integer multiple of a distance (angle) between adjacent position adjustment protrusions 262. That is, when the lower case 24 is located at the zero-point, the position adjustment sensor 110 is arranged to face an end of one or more position adjustment protrusions 262.

In the fluid distribution system 1 according to the present disclosure, it may be checked whether the second transfer hole 244 faces one third upper hole of the plurality of third upper holes 228 by using information sensed by the position adjustment sensor 110 and the zero-point adjustment sensor 120, and a third upper hole through which the fluid flowing into the selector 20 is to be discharged, may be selected.

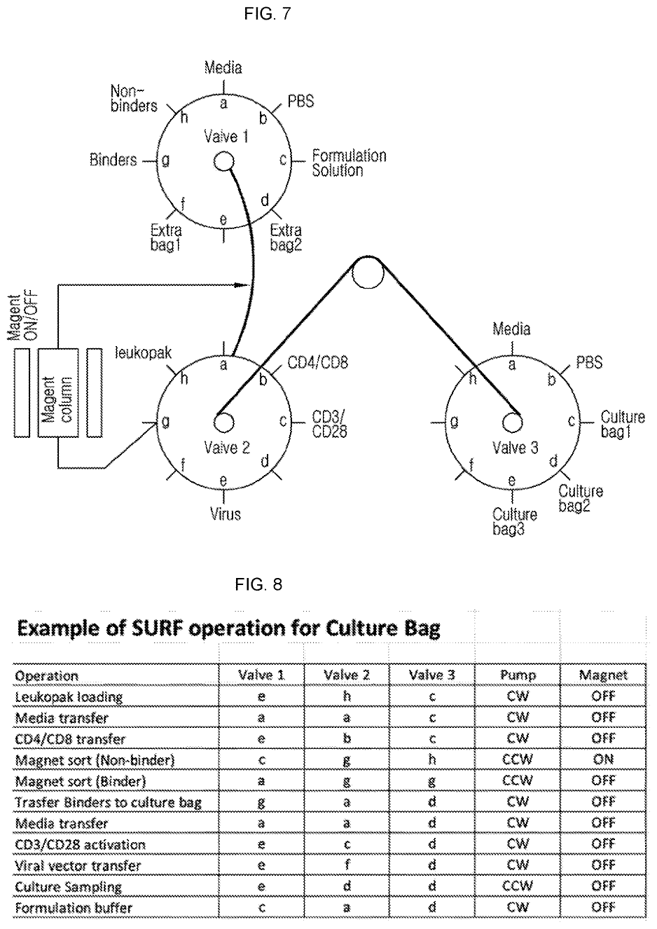

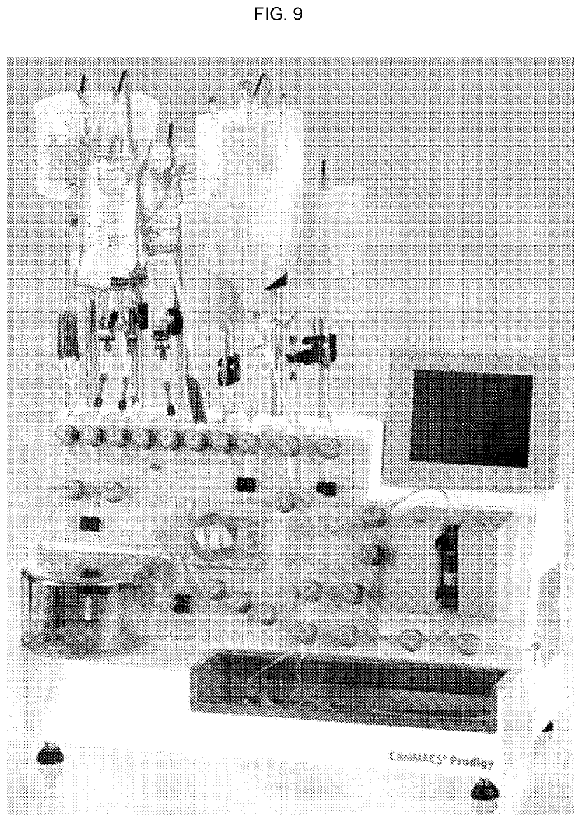

FIGS. 7 and 8 illustrate a method of operating a fluid distribution system according to another embodiment of the present disclosure.

Piping and instrument diagram (P & ID) shown in FIG. 7 represents a fluid distribution system in which eight selectors according to an embodiment are arranged. In FIG. 7, Valve 1 to Valve 3 are one selector, and letters indicated around each selector are used to distinguish a plurality of third upper holes from each other.

Meanwhile, a magnet column shown in FIG. 7 represents a check valve (or one-way valve. The magnet column may be used to sort specific types of cells, such as CD4 or CD6 positive T cells. When the magnet column is on, CD4 or CD6 positive T cells are transferred to a third upper hole h of a selector Valve 1, and when the magnet column is off, CD4 or CD6 positive T cells are transferred to a third upper hole g of the selector Valve 1.

In an example, the case where leukopak is loaded, will be described. Referring to FIG. 7, nothing is connected to the third upper hole e of the selector Valve 1. Thus, the magnet column is off.

Leukopak is connected to the third upper hole h of the selector Valve 2. In this case, leukopak flowing into the selector Valve 2 through the third upper hole h is discharged to the conduit through a first port.

Leukopak flowing through a conduit connecting the selector Valve 2 to the selector Valve 3 may flow into a first upper hole of the selector Valve 3) and may be transferred to Culture bag 1 connected to the third upper hole c of the selector Valve 3.

In this case, rotation of each selector may be properly controlled by electrical signals applied to the motor connected to each selector, and these electrical control signals may be directly input by the user through the manipulation portion 140. Alternatively, the electrical control signals may be signals generated by using information previously stored in a controller (not shown).

An operating method according to all scenarios described in FIG. 8 is performed in a similar operating method to the above description, and detailed descriptions thereof will be omitted.

The above description is merely an illustrative explanation of the technical idea of this embodiment, and those skilled in the art will be able to make various modifications and variations without departing from the essential characteristics of this embodiment. Accordingly, the present embodiments are not intended to limit the technical idea of the present embodiment, but rather to explain it, and the scope of the technical idea of the present embodiment is not limited by these examples. The scope of protection of this embodiment should be interpreted in accordance with the claims below, and all technical ideas within the equivalent scope should be interpreted as being included in the scope of rights of this embodiment.

### EXPLANATION OF REFERENCE NUMERALS

1: fluid distribution system
10: main body
20: selector
22: upper case
24: lower case
26: main body coupling portion
30: main body cover
40: lines

1. A fluid distribution system comprising:
a main body;
a plate-shaped main body cover formed to cover at least part of an upper portion of the main body; and
a plurality of selectors arranged on the main body cover to be spaced apart from each other and to make fluid communication with each other,
wherein one selector comprises:
a cylindrical upper case comprising a first port extending from a center of an upper surface to an outer circumferential surface of the upper case on the upper surface and a plurality of third upper holes arranged along the outer circumferential surface of the upper case at equal intervals and having an open lower surface;
a cylindrical lower case having at least part inserted into the upper case through the lower surface of the upper case; and
a main body coupling portion protruding downwards from a bottom surface of the lower case and coupled to the main body.

2. The fluid distribution system of claim 1, wherein the upper case comprises:
a first upper hole formed on one surface of the first port formed in parallel to an outer circumferential surface of the upper case;
a second upper hole formed in a center of the upper surface of the upper case and passing through an interior ceiling surface of the upper case from the upper surface of the upper case; and
an upper flow path extending from the first upper hole to the second upper hole and formed inside the first port.

3. The fluid distribution system of claim 2, wherein the lower case comprises:
a first transfer hole formed in an upper surface of the lower case and facing the second upper hole;
a second transfer hole formed in an outer circumferential surface of the lower case and facing one of the plurality of third upper holes; and
a transfer flow path extending from the first transfer hole to the second transfer hole and formed inside the lower case.

4. The fluid distribution system of claim 3, wherein the main body coupling portion comprises:
a coupling groove formed toward an inside from a bottom surface of the main body coupling portion;
a position adjustment protrusion arranged on an outer circumferential surface of the main body coupling portion; and
a zero-point adjustment protrusion arranged on the outer circumferential surface of the main body coupling portion,
wherein a distance between the bottom surface of the main body coupling portion and an end of the position adjustment protrusion is greater than a distance between the bottom surface of the main body coupling portion and an end of the zero-point adjustment protrusion.

5. The fluid distribution system of claim 5, wherein a number of the plurality of third upper holes is n (where n is a natural number), one zero-point adjustment protrusion is arranged, and (n−1) position adjustment protrusions are arranged, and the zero-point adjustment protrusion and the position adjustment protrusion are arranged at equal intervals.

6. The fluid distribution system of claim 5, wherein a center of the zero-point adjustment protrusion is aligned with a center of the second transfer hole.

7. The fluid distribution system of claim 6, wherein the main body comprises:
a selector installation portion into which the main body coupling portion is inserted;
a power transmission portion formed in a center of the selector installation portion and inserted into the coupling groove;
a zero-point adjustment sensor arranged on an inner circumferential surface of the selector installation portion and formed to face one end of the zero-point adjustment protrusion; and
a position adjustment sensor arranged on the inner circumferential surface of the selector installation portion and formed to face one end of one of the position adjustment protrusions.

8. The fluid distribution system of claim 1, wherein the main body comprises:
a motor arranged inside of the main body and configured to rotate the main body coupling portion; and
a manipulation portion configured to adjust a rotation angle and rotation speed of the motor.

9. The fluid distribution system of claim 1, wherein the main body cover comprises a magnet, and the main body further comprises a metal material coupled to the magnet.

10. The fluid distribution system of claim 1, wherein the main body further comprises a plurality of guide shafts arranged on an upper surface of the main body, and the main body cover comprises a plurality of guide holes through which the plurality of guide shafts pass so that the main body cover is coupled to the main body in a preset position.

11. The fluid distribution system of claim 1, further comprising lines configured to connect at least one of a plurality of third upper holes of one among the plurality of selectors to a first upper hole of another one among the plurality of selectors.

12. The fluid distribution system of claim 11, further comprising a pump, wherein the lines are connected to the pump.
